(19) United States
(12) Patent Application Publication
(10) Pub. No.: US 2020/0093100 A1
(43) Pub. Date: Mar. 26, 2020

(54) A DATA COLLECTION SYSTEM AND METHOD FOR COLLECTING DATA RELATING TO THE BEHAVIOUR OF AN ANIMAL

(71) Applicant: DAIRYMASTER, County Kerry (IE)

(72) Inventors: Edmond Patrick HARTY, County Kerry (IE); John Gerard DALY, County Kerry (IE)

(73) Assignee: DAIRYMASTER, County Kerry (IE)

(21) Appl. No.: 16/604,799

(22) PCT Filed: Apr. 13, 2018
(86) PCT No.: PCT/IE2018/000004

§ 371 Date: Oct. 11, 2019

(30) Foreign Application Priority Data
Apr. 13, 2017 (IE) S2017/0083

(51) Int. Cl. A01K 29/00; B64C 39/02; A01K 11/00; H02J 7/35; H04N 7/18; G06K 9/00; H04W 4/029; H04W 4/44; A61B 5/0205

(52) U.S. Cl. CPC A01K 29/005; B64C 39/02; A01K 11/004; H02J 7/35; H04N 7/183; G06K 9/00362; B64F 1/362; H04N 7/181; H04W 4/029; H04W 4/44; A61B 5/02055; B64C 2201/123; G06K 9/00335

(57) ABSTRACT

A data collection system (1) for monitoring the behaviour and states of a plurality of animals (2) comprises providing the animals (2) with respective monitoring devices (4) for monitoring the behaviour and states of the animals (2). A drone (7) comprising a mobile data collection device (5) secured thereto is configured to fly past the animals (2) at predefined data collection time intervals, typically of six hourly intervals. Each mobile data collection device (5) comprises a first secondary communications module (15) for wirelessly communicating with a second secondary communications module (32) located in the respective monitoring devices (4) for uploading data indicative of the behaviour and states of the animals (2) to a microcontroller (8) in the mobile data collection device (5). The uploaded data indicative of the behaviour and states of the respective animals (2) is written to and stored in a primary memory (17) of the mobile data collection device (5). A primary communications module (10) in the mobile data collection device (5) transmits the stored data indicative of the behaviour and states of the respective animals (2) which is stored in the primary memory (17) wirelessly to a central server (12). In an alternative embodiment of the invention the mobile data collection device (5) is mounted on one of the animals (2). In a further alternative embodiment of the invention the monitoring devices (4) are omitted from the animals, and the mobile data collection device is provided with a video camera for capturing images and video clips of the respective animals, and data from the video camera is analysed for identifying the animals and the behaviour and states of the respective animals.

The present invention relates to a data collection system for collecting data relating to at least one of the behaviour, at least one state and at least one characteristic of a plurality of animals. The invention also relates to a method for collecting data relating to at least one of the behaviour, at least one state and at least one characteristic of a plurality of animals.

In some farms, particularly in farms where animals are remotely located relative to the main part of the farm, it can be difficult to monitor remotely located animals in order to determine the behaviour, state and characteristics of such animals, for example, it is important to a farmer to know at reasonably regular intervals the behaviour, states and characteristics of animals. For example, it is important to know the activity level of an animal. For example, if an animal is in a state of relatively high activity, this could be an indication of the animal being in heat. If an animal is in a relatively low state of activity, this could be an indication of an animal being unwell, or having an injury, particularly, an injury to a leg or foot of the animal. It is also advantageous for a farmer to know periodically the temperature, heart rate, blood pressure and the like of an animal in order to ascertain the health state of the animal.

In fragmented farms, where a farm does not comprise a single area of land, but rather a number of unconnected areas of land, which are commonly referred to a “out farms”, farmers tend to move the animals from one out farm to another at periodic intervals, for example, a herd of animals, such as a herd of suckler cows may be moved from one out farm to another out farm after a period of two or three days. Such out farms may be a considerable distance apart from each other, and may be well over two kilometres apart. Additionally, such out farms may be a considerable distance from the main farm, and may also be well over two kilometres from the main farm. In such cases, in general, it is not feasible for a farmer to regularly monitor the behaviour, states or characteristics of animals in such out farms.

In general, the only feasible way to monitor animals in such out farms, is to provide the animals with monitoring devices, of the type which are disclosed in PCT Published Application Specification No's. WO2014/199361; WO2014/199362 and WO2014/199363. However, in general due to the distance of such out farms from the main farm, it is not feasible to transmit data indicative of the behaviour, states and characteristics of animals from such respective monitoring devices, due to the high level of battery power required to transmit the relevant data over such distances to the main farm. This, in general, requires the location of base stations or data collection devices to be located at strategic locations in each of the out farms. In many such out farms, mains electricity is unavailable, and thus, such base stations or data collection devices must be powered by batteries. Due to the relatively high power consumption required by such intermediate base stations to transmit data collected from the monitoring devices to, for example, a central server on the main farm, the batteries of such intermediate base stations require regular replacement or recharging. This, is inconvenient, and additionally, the cost of providing such intermediate base stations is relatively high.

There is therefore a need for a data collection system for collecting data indicative of at least one of the behaviour, at least one state and at least one characteristic of a plurality of animals, which addresses these problems. There is also a need for a method for collecting data indicative to at least one of the behaviour, at least one state and at least one characteristic of a plurality of animals.

The present invention is directed towards providing such a data collection system and such a method.

According to the the invention there is provided a method for monitoring at least one of the behaviour, at least one state and at least one characteristic of each of a plurality of animals, the method comprising acquiring data indicative of the at least one of the behaviour, the at least one state and the at least one characteristic of each of the animals by a mobile data collection device, storing the acquired data in a primary storing means of the mobile data collection device, communicating the acquired data stored in the primary storing means of the mobile data collection device to a central server, and mounting the mobile data collection device on one of a prime mover and one of the animals.

In one aspect of the invention the data indicative of the at least one of the behaviour, the at least one state and the at least one characteristic of the animals is acquired by the mobile data collection device by a data acquisition means.

In another aspect of the invention the data acquisition means comprises an imaging means.

In a further aspect of the invention the data acquisition means of the mobile data collection device comprises an imaging device.

In a still further the data acquisition means comprises one of a camera and a video camera.

In one aspect of the invention the data acquisition means comprises a charge coupled imaging device.

Preferably, data indicative of the identity of each of the animals is acquired by the mobile data collection device.

Advantageously, the data indicative of the identity of each animal is stored and cross-referenced with the determined one of the behaviour, the at least one state and the at least one characteristic of the animal in the primary storing means of the mobile data collection device.

Preferably, the data indicative of the identity of each of the animals is determined from at least one of an image of the animal captured by the imaging means.

Advantageously, the identity of each animal is determined from a plurality of images sequentially captured by the imaging means.

In another aspect of the invention the identity of each animal is determined by an image recognition means configured for identifying the respective animals from respective images thereof captured by the imaging means.

Preferably, the image recognition means is located in the mobile data collection device.

In another aspect of the invention the at least one of the behaviour, the at least one state and the at least one characteristic of each animal is determined from one or more images of the animal captured by the imaging means.

In a further aspect of the invention the at least one of the behaviour, the at least one state and the at least one characteristic of each animal is determined from a series of images of the animal sequentially captured by the imaging means.

In one aspect of the invention the images captured by the imaging means are analysed for determining the at least one of the behaviour, the at least one state and the at least one characteristic of the animal.

In another aspect of the invention a series of the images captured by the imaging means of the mobile data collection device are analysed for determining the at least one of the behaviour, the at least one state and the at least one characteristic of a corresponding one of the animals.

In a further aspect of the invention each animal is provided with an identifying means comprising data indicative of the identity of the corresponding animal.

Preferably, the identifying means of each animal comprises the data indicative of the identity of the animal in the form of an electronically readable identifying code.

Alternatively or additionally, the identifying means of each animal comprises a vision readable identity code.

Advantageously, identifying code is read from the identifying means by the data acquisition means of the mobile data collection device.

Preferably, the identifying means is located one of externally on and internally in the animal.

In one aspect of the invention the mobile data collection device comprises a location tracking means.

Preferably, the location tracking means cooperates with a GPS network for identifying the location of the mobile data collection device.

Advantageously, the location of each animal is determined by the location tracking means of the mobile data collection device.

Preferably, the mobile data collection device communicates data indicative of the location of each animal cross-referenced with the data indicative of the identity of the animal to the central server.

In one aspect of the invention the mobile data collection device communicates the data indicative of the one of the behaviour, the at least one state and the at least one characteristic of each animal cross-referenced with the identity of the animal to the central server wirelessly.

Preferably, the mobile data collection device communicates with the central server via a wireless communications network.

Advantageously, the mobile data collection device communicates with the central server via a telecommunications network.

Preferably, the mobile data collection device communicates with the central server via a GMS communications network.

In another aspect of the invention the data indicative of the at least one of the behaviour, the at least one state and the at least one characteristic of each animal is communicated to the central server by the mobile data collection device just after it has been stored in the primary storing means of the mobile data collection device.

In a further aspect of the invention the mobile data collection device is mounted on one of the animals.

In one aspect of the invention the data indicative of the at least one of the behaviour, the at least one state and the at least one characteristic of each animal is communicated to the central server by the mobile data collection device at predefined transmission time intervals. Preferably, each predefined transmission time interval is of time duration in the range of 3 hours to 24 hours. Advantageously, each predefined transmission time interval is of time duration in the range of 4 hours to 12 hours. Ideally, each predefined transmission time interval is of time duration of approximately 6 hours.

In one aspect of the invention the mobile data collection device is mounted on the prime mover and is urged past the animals by the prime mover at predefined data collection time intervals.

Preferably, the time duration of each predefined data collection time interval lies in the range of 3 hours to 24 hours. Preferably, the time duration of each predefined data collection time interval lies in the range of 4 hours to 12 hours. Advantageously, the time duration of each predefined data collection time interval is approximately 6 hours.

In another aspect of the invention the mobile data collection device is mounted on the prime mover, so that the mobile data collection device is urged past the respective animals within a distance of up to 10 metres of each animal.

In another aspect of the invention the mobile data collection device is mounted on the prime mover, so that the mobile data collection device is urged past the respective animals within a distance of up to 7 metres of each animal.

Preferably, the mobile data collection device is mounted on the prime mover, so that the mobile data collection device is urged past the respective animals within a distance of approximately 3 metres of each animal.

In one aspect of the invention the mobile data collection device is mounted on the prime mover in the form of one of a drone configured for flying, a tractor and a quad-bike.

In a further aspect of the invention the data acquired by the mobile data collection device is communicated to the central server on completion of each pass of the animals by the mobile data collection device.

In another aspect of the invention the data indicative of the at least one of the behaviour, the at least one state and the at least one characteristic of each animal acquired by the mobile data collection device is communicated by the mobile data collection device to the central server by a hard wire connection.

In another aspect of the invention each animal is provided with a monitoring device configured for monitoring the at least one of the behaviour, the at least one state and the at least one characteristic of the corresponding animal.

In one aspect of the invention each monitoring device is configured for monitoring the at least one of the behaviour, the at least one state and the at least one characteristic of the corresponding animal during predefined monitoring time periods, and at the end of each predefined monitoring time period data captured during that predefined monitoring time period indicative of the at least one of the behaviour, the at least one state and the at least one characteristic is stored. Preferably, the time duration of each predefined monitoring time period lies in the range of 5 minutes to 60 minutes. Advantageously, the time duration of each predefined monitoring time period lies in the range of 10 minutes to 45 minutes. Preferably, the time duration of each predefined monitoring time period is approximately 15 minutes.

Advantageously, the predefined monitoring time period run consecutively one after the other.

In another aspect of the invention the identifying means comprising the identity of each animal is located in the monitoring device of the corresponding animal.

Preferably, each monitoring device comprises a secondary storing means configured for storing data indicative of the at least one of the behaviour, the at least one state and the at least one characteristic of the corresponding animal cross referenced with the identity of the animal.

Preferably, the data stored in the secondary storing means of each monitoring device is acquired from the monitoring devices by the data acquisition means of the mobile data collection device.

Advantageously, the data stored in the secondary storing means of each monitoring device is acquired wirelessly by the data acquisition means of the mobile data collection device.

In one aspect of the invention the data stored in the secondary storing means of each monitoring device is acquired wirelessly by the data acquisition means of the mobile data collection device by radio frequency protocol transmission.

Preferably, the data stored in the secondary storing means of each monitoring device is acquired wirelessly by the data acquisition means of the mobile data collection device by passive communication, under which the monitoring device is powered wirelessly by the mobile data collection device.

In one aspect of the invention the data stored in the secondary storing means of each monitoring device is acquired by the data acquisition means of the mobile data collection device by RFID communications.

In a further aspect of the invention the data stored in the secondary storing means of each monitoring device is acquired by the data acquisition means of the mobile data collection device by passive RFID communications.

Preferably, each monitoring device is configured for mounting one of externally on and internally in the corresponding animal.

Preferably, each monitoring device is activated by a signal from the mobile data collection device to initiate acquisition of data from the storing means in the corresponding mobile device by the mobile data collection device.

In one aspect of the invention at least one of the behaviour, the at least one state and the at least one characteristic of the animal relates to one or more of resting, feeding, ruminating and the level of activity of the animal.

In another aspect of the invention the level of activity monitored of each animal is categorised as being one of a highly active state, a medium active state and a low active state.

Preferably, the level of activity monitored of each animal is categorised as being one of a medium active state, the medium active state being of activity less than the highly active state, and activity greater than the low active state.

In a further aspect of the invention the at least one characteristic of each animal which is monitored comprises one of body temperature, blood pressure and heart rate.

In one aspect of the invention the data indicative of the at least one of the behaviour, the at least one state and the at least one characteristic of each animal is communicated to the central server by the mobile data collection device in response to receipt of a signal from the central server.

In another aspect of the invention the data indicative of the one of the behaviour, the at least one state and the at least one characteristic of one or more of the animals is communicated to the central server by the mobile collection device in response to the data indicative of the one of the behaviour, the at least one state and the at least one characteristic being indicative of an emergency state being detected in that one or more of the animals.

In one aspect of the invention the animals are remotely located.

The invention also provides a data collection system comprising a mobile data collection device configured for carrying out the method according to the invention for monitoring at least one of the behaviour, at least one state and at least one characteristic of each of a plurality of animals.

Additionally the invention provides a data collection system for collecting data relating to at least one of the behaviour, at least one state and at least one characteristic of a plurality of animals, the system comprising a mobile data collection device, the mobile data collection device comprising a data acquisition means for acquiring data indicative of the at least one of the behaviour, the at least one state, and the at least one characteristic of each animal, a primary storing means for storing the acquired data, and a primary communicating means for communicating the stored data in the primary storing means to a central server, the data collection device being configured for mounting on one of a prime mover and one of the animals.

In one aspect of the invention the data acquisition means comprises an imaging means.

In another aspect of the invention the data acquisition means comprises an imaging device.

In another aspect of the invention the data acquisition means comprises one of a camera and a video camera.

In another aspect of the invention the data acquisition means comprises a charged coupled imaging device.

Preferably, the mobile data collection device comprises an image recognition means configured for identifying each animal from at least one image of the animal captured by the imaging means.

In one aspect of the invention the image recognition means is configured for identifying each animal from a series of images of the animal captured by the imaging means.

Preferably, the image recognition means is configured for identifying each animal from a series of images of the animal sequentially captured by the imaging means.

In another aspect of the invention the mobile data collection device comprises an analysing means for analysing at least one image of each animal captured by the imaging means for determining the at least one of the behaviour, the at least one state and the at least one characteristic of the animal.

Preferably, the analysing means is configured for analysing a series of images of each animal captured by the imaging means for determining the at least one of the behaviour, the at least one state and the at least one characteristic of the animal.

Advantageously, the analysing means is configured for analysing a series of images of each animal sequentially captured by the imaging means for determining the at least one of the behaviour, the at least one state and the at least one characteristic of the animal.

In one aspect of the invention a plurality of identifying means are provided, one identifying means being provided for each animal, and each identifying means comprising data indicative of the identity of the corresponding animal, and the data acquisition means is configured to read the data indicative of the identity of the animals from the respective identifying means.

In another aspect of the invention each identifying means comprises an electronically readable identity code identifying the corresponding animal.

Alternatively or additionally, each identifying means comprises a vision readable identity code identifying the corresponding animal.

In a further aspect of the invention the data acquisition means is configured for reading the identity code from each of the identifying means.

Preferably, each identifying means is configured for one of locating externally on and internally in the corresponding animal.

In another aspect of the invention the primary storing means of the mobile data collection device is configured for storing data indicative of the one of the behaviour, the at least one state and the at least one characteristic of each animal cross-referenced with the identity of the animal.

In one aspect of the invention the mobile data collection device comprises a location tracking means.

Preferably, the location tracking means of the mobile data collection device cooperates with a GPS network in order to identify the location of the mobile data collection device.

In another aspect of the invention the mobile data collection device is configured for determining the location of each animal from the location of the mobile data collection device.

Preferably, the mobile data collection device is configured to store data indicative of the location of each animal cross-referenced with the data indicative of the identity of the animal in the primary storing means.

In another aspect of the invention the primary communicating means of the mobile data collection device is configured to wirelessly communicate with the central server for transmitting the data stored in the primary storing means thereof indicative of the at least one of the behaviour, the at least one state, the at least one characteristic of each animal and the location of each animal cross-referenced with the identity data of the animal.

In another aspect of the invention the primary communicating means of the mobile data collection device is configured to communicate the data stored in the primary storing means thereof to the central server via a wireless communications network.

In a further aspect of the invention the primary communicating means of the mobile data collection device is configured to communicate the data stored in the primary storing means thereof to the central server via a GSM network.

In another aspect of the invention the primary communicating means of the mobile data collection device is configured for communicating the stored data in the primary storing means thereof to the central server just after the collection of the stored data.

Preferably, the primary communicating means of the mobile data collection device is configured for uploading the stored data in the primary storing means thereof to the central server at predefined transmission time intervals.

Advantageously, the time duration of each predefined transmission time interval lies in the range of 3 hours to 24 hours.

Preferably, the time duration of each predefined transmission time interval lies in the range of 4 hours to 12 hours.

Advantageously, the time duration of each predefined transmission time interval is approximately 6 hours.

In another aspect of the invention the data indicative of the at least one of the behaviour, the at least one state and the at least one characteristic of each animal stored in the primary storing means of the mobile data collection device is communicated to the central server by the mobile data collection device in response to receipt of a signal from the central server requesting uploading of the stored data.

In a further aspect of the invention the data indicative of the one of the behaviour, the at least one state and the at least one characteristic of one or more of the animals is communicated to the central server in response to the data indicative of the one of the behaviour, the at least one state and the at least one characteristic being indicative of an emergency state being detected in that of one or more of the animals.

In one aspect of the invention the mobile data collection device is powered by an electrical battery. Preferably, the electrical battery of the mobile data collection device comprises a rechargeable battery.

Preferably, the mobile data collection device comprises a solar panel for powering the mobile data collection device and for charging the electrical battery thereof. Advantageously, the solar panel of the mobile data collection device comprises a photovoltaic panel.

Alternatively, the mobile data collection device is configured to be powered by the prime mover on which the mobile data collection device is mounted.

In one aspect of the invention the mobile data collection device is configured for mounting on one of the animals.

In another aspect of the invention the mobile data collection device is configured for mounting on the prime mover, and is configured to be urged past the animals by the prime mover at predefined data collection time intervals.

In another aspect of the invention the time duration of each predefined data collection time interval lies in the range of 3 hours to 24 hours.

In a further aspect of the invention the time duration of each predefined data collection time interval lies in the range of 4 hours to 12 hours.

In a still further aspect of the invention the time duration of each predefined data collection time interval is approximately 6 hours.

In one aspect of the invention when the mobile data collection device is configured for mounting on a prime mover, the primary communicating means is configured for uploading the data stored in the primary storing means indicative of the at least one of the behaviour, the at least one state, the at least one characteristic and the location of each animal cross-referenced with the identity thereof to the central server when the mobile data collection device has returned from each pass of the animals. Preferably, a docking station configured to communicate with the central server is provided and the mobile data collection device is configured to dock in the docking station, and to communicate the data stored in the primary storing means thereof indicative of the at least one of the behaviour, the at least one state, the at least one characteristic and the location of each animal cross-referenced with the identity thereof to the central server via the docking station when the mobile data collection device is docked in the docking station.

Advantageously, the mobile data collection device is configured to be mounted on the prime mover so that the mobile data collection device passes within a distance of up to 10 metres from each of the animals.

In another embodiment of the invention the mobile data collection device is configured to be mounted on the prime mover so that the mobile data collection device passes within a distance of up to 7 metres from each of the animals.

Preferably, the mobile data collection device is configured to be mounted on the prime mover so that the mobile data collection device passes within a distance of approximately 3 metres from each of the animals.

In another aspect of the invention the mobile data collection device is configured for mounting on a prime mover in the form of one of a tractor, a quad-bike and a drone configured for flying.

Preferably, the prime mover comprises an autonomous vehicle.

In another aspect of the invention a plurality of monitoring devices are provided, one monitoring device being provided for each animal, and being configured for attaching to the corresponding animal, each monitoring device being configured for monitoring the at least one of the behaviour, the at least one state and the at least one characteristic of the corresponding animal, and each monitoring device comprising a secondary storing means for storing data indicative of the at least one of the behaviour, the at least one state and the at least one characteristic of the animal.

Preferably, each monitoring device is configured for locating one of externally on and internally in the corresponding animal.

Advantageously, the data acquisition means of the mobile data collection device comprises a first secondary communicating means for wirelessly communicating with each of the monitoring devices for receiving the stored monitored data therefrom.

Preferably, each monitoring device comprises a second secondary communicating means configured for wirelessly transmitting data stored in the secondary storing means of the monitoring device for reception by the mobile data collection device.

In one aspect of the invention the first secondary communicating means of the mobile data collection device is configured for communicating wirelessly with the second secondary communicating means of each monitoring device.

In another aspect of the invention the first secondary communicating means of the mobile data collection device is configured for communicating wirelessly with the second secondary communicating means of each monitoring device by a radio frequency protocol transmission.

Preferably, the first and second secondary communicating means comprise RFID communicating means.

In one aspect of the invention the RFID communicating means of each monitoring device comprises a passive RFID communicating means.

In another aspect of the invention the second secondary communicating means of each monitoring device is configured for powering by the mobile data collection device.

Preferably, the second secondary communicating means of each monitoring device is configured to be powered by electromagnetic induction by a signal transmitted by the first secondary communicating means of the mobile data collection device.

In one aspect of the invention each monitoring device is configured to transmit data in response to an activating signal from the mobile data collection device.

In another aspect of the invention each monitoring device is configured to upload data from the secondary storing means thereof to the mobile data collection device through the second secondary communicating means of the monitoring device and the first secondary communicating means of the mobile data collection device.

Preferably, the primary storing means of the mobile data collection device is configured for storing data uploaded from the respective monitoring devices.

Advantageously, each monitoring device comprises the corresponding one of the identifying means.

In one aspect of the invention each monitoring device is configured for monitoring the at least one of the behaviour, the at least one state and the at least one characteristic of the corresponding animal during predefined monitoring time periods, and at the end of each predefined monitoring time period, the data captured during that predefined monitoring time period indicative of the at least one of the behaviour, the at least one state and the at least one characteristic of the animal is stored in the secondary storing means. Preferably, the time duration of each predefined monitoring time period lies in the range of 5 minutes to 60 minutes. Advantageously, the time duration of each predefined monitoring time period lies in the range of 10 minutes to 45 minutes. Ideally, the time duration of each predefined monitoring time period is approximately 15 minutes.

Advantageously, the predefined monitoring time periods run consecutively one after the other.

In one aspect of the invention each monitoring device is powered by an electrical battery. Preferably, the electrical battery of each monitoring device comprises a rechargeable battery.

In one aspect of the invention the mobile data collection device comprises a smart mobile device.

Preferably, the mobile data collection device comprises a smart mobile phone.

In one aspect of the invention the animals are remotely located.

The advantages of the invention are many. A particularly important advantage of the invention is that the data collection system according to the invention is particularly suitable for collecting data indicative of at least one of the behaviour, at least one state and at least one characteristic of an animal, for example, each animal of a herd of animals, and in particular animals which are in a remote location, for example, on an out farm outlying some considerable distance from the main farm.

The advantage of providing the mobile data collection device of the data collection system with an imaging means provides a particularly important advantage, in that only one mobile data collection device is required for monitoring the behaviour, states and/or characteristics of a plurality of animals, and there is no need to provide the other animals with any other monitoring devices, since all monitoring of the animals is carried out by the mobile data collection device.

A particularly important advantage of the invention is achieved when the mobile data collection device is mounted on one of the animals of a herd of animals, in that the mobile data collection device moves with the herd of the animals, since the animal on which the mobile data collection device is mounted moves with the herd.

The invention will be more clearly understood from the following description of some preferred embodiments thereof which are given by way of example only with reference to the the accompanying drawings in which:

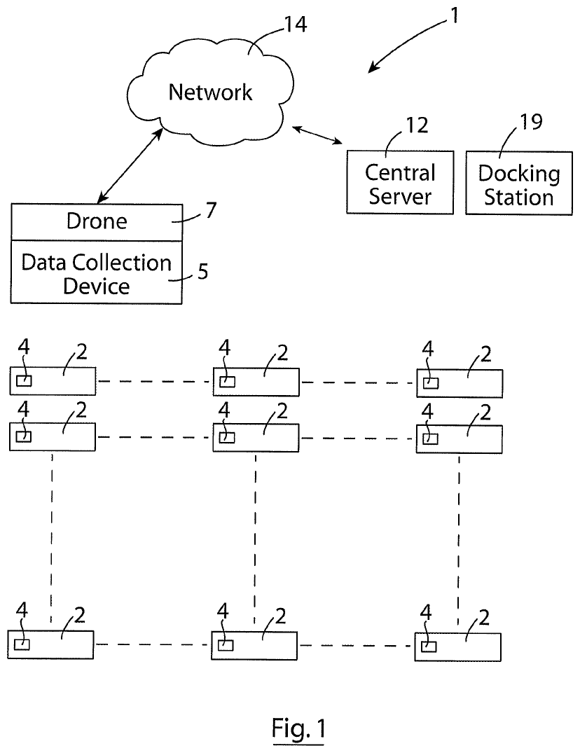

FIG. 1 is a block representation of a data collection system according to the invention for monitoring and collecting data indicative of the behaviour, the states and/or characteristics of a plurality of animals,

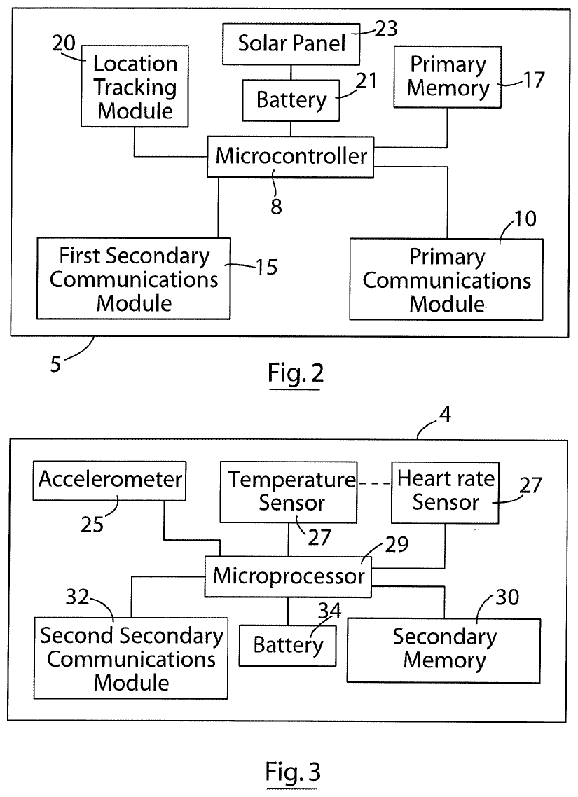

FIG. 2 is a block representation of a mobile data collection device of the system of FIG. 1,

FIG. 3 is a block representation of one of a plurality of monitoring devices of the system of FIG. 1,

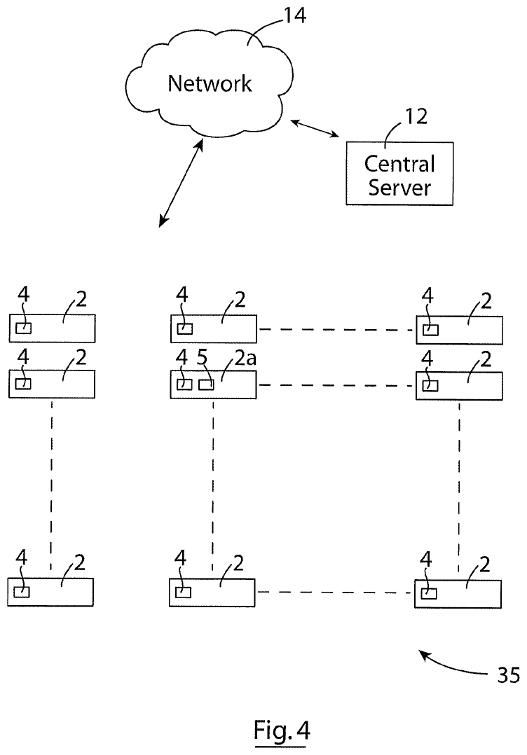

FIG. 4 is a block representation of a data collection system according to another embodiment of the invention for monitoring and collecting data indicative of the behaviour, states and/or characteristics of a plurality of animals,

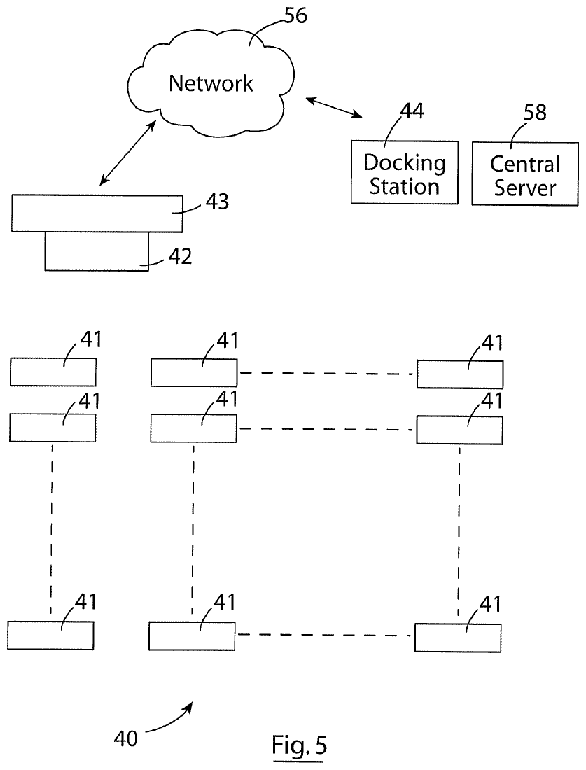

FIG. 5 is a block representation of a data collection system according to another embodiment of the invention for monitoring and collecting data indicative of the behaviour, the states and/or characteristics of a plurality of animals,

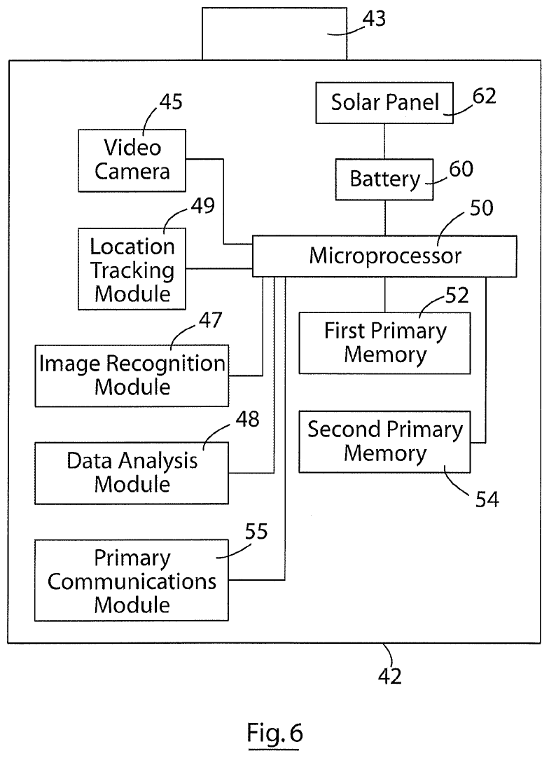

FIG. 6 is a block representation of a mobile data collection device of the system of FIG. 5,

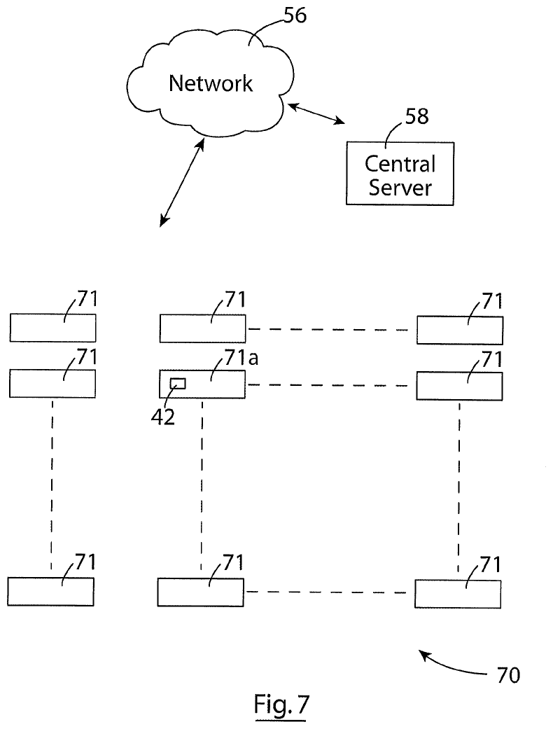

FIG. 7 is a block representation of a data collection system according to a further embodiment of the invention for monitoring and collecting data indicative of the behaviour, the states and/or characteristics of a plurality of animals, and

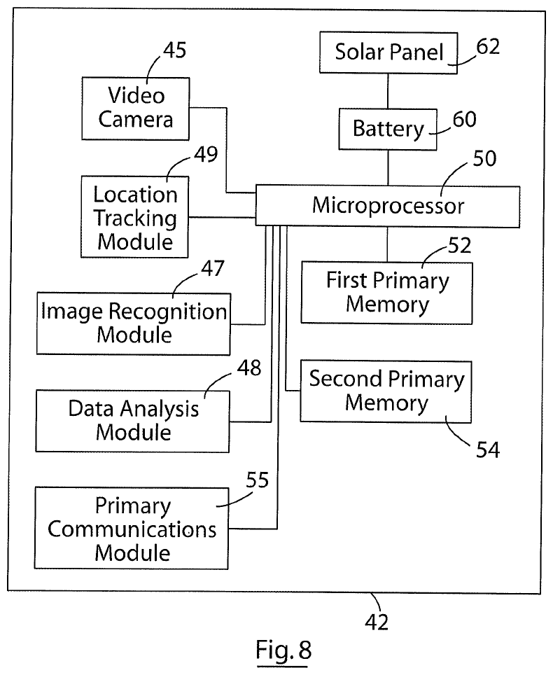

FIG. 8 is a block representation of a mobile data collection device of the system of FIG. 7.

Referring to the drawings and initially to FIGS. 1 to 3 thereof, there is illustrated a mobile data collection system according to the invention indicated generally by the reference numeral 1 for monitoring at least one of the behaviour, at least one state and at least one characteristic of a plurality of animals 2 and for collecting data indicative of the at least one of the behaviour, the at least one state and the at least one characteristic of the respective animals 2. The animals 2 are illustrated in block representation, and may be of any type of animal, the behaviour, the state or states or a characteristic or characteristics thereof, of which are to be monitored. In this embodiment of the invention the animals 2 are suckler cows, but they may be any type of cows, cattle, or any other animals. Additionally, in this embodiment of the invention the animals 2 are in an area which is remotely located relative to the main farm, and are in a field or fields which may be a number of kilometres away from the main farm. However, it will be appreciated that the animals may be located in any suitable location, and in some cases may be located in a field or fields located in the main farm, or may be housed in an animal house on the main farm or remotely located therefrom.

The system 1 comprises a plurality of monitoring devices 4, one monitoring device 4 being provided for each animal 2. The monitoring devices 4 are configured for monitoring the behaviour, one or more states and/or characteristics of the respective animals 2, in order to determine one or more of the behaviour, the states and/or characteristics of the respective animals 2, including, for example, resting, feeding, ruminating, as well as the level of activity of the animals 2, for example, whether any one or more of the animals 2 are in a highly active state, which could be indicative of the animal being in heat, a medium active state or a relatively low active state, which could be indicative of the animal being unwell or injured. The monitoring devices 4 may also be configured to monitor other characteristics of the animals, for example, the temperature of the respective animals, the blood pressure of the respective animals, the heart rate of the animals and other such characteristics. Data indicative of the monitored behaviour, states and/or characteristics of each animal 2 is stored in the corresponding monitoring device 4 for subsequent collection, as will be described below. Such monitoring devices as the monitoring devices 4 are disclosed in PCT Specification Nos. WO 2014/199361; WO 2014/199362 and WO 2014/199363. The monitoring devices 4 will be described in further detail below.

The system 1 also comprises a mobile data collection device 5 for communicating sequentially with the respective monitoring devices 4, and for sequentially collecting stored data from the monitoring devices 4. In this embodiment of the invention the mobile data collection device 5 is configured for mounting on a prime mover, in this case a drone 7. The drone 7 is of the type which incorporates autonomous flying technology, and its flight path is programmable so that the flight path of the drone 7 may be programmed to fly past the animals 2 at predefined data collection time intervals for uploading the stored data from the monitoring devices 4. In this embodiment of the invention each predefined data collection time interval is selectable and may, for example, be six, twelve or twenty-four hourly intervals, or longer or shorter, depending on the frequency with which it is desired that the stored data be collected from the monitoring devices 4.

In this embodiment of the invention the mobile data collection device 5 comprises a primary control means, namely, a microcontroller 8 which controls the operation of the mobile data collection device 5. The data collection device 5 comprises a primary communicating means, in this embodiment of the invention a primary two-way data communications module 10 for wirelessly communicating with a central server 12 of the data collection system 1 for transmitting data collected from the monitoring devices 4 to the central server 12 via a wireless communications network 14, for example, a GSM network, the Ethernet or any other suitable wireless communications network.

A data acquisition means, which in this embodiment of the invention comprises a first secondary communicating means is provided in the mobile data collection device 5 and comprises a first secondary communications module 15 for wirelessly communicating with the monitoring devices 4 for uploading data therefrom indicative of the data indicative of the behaviour and the various states and characteristics of the corresponding animals 2 cross-referenced with the identities of the respective animals 2 to the mobile data collection device 5. In this embodiment of the invention the first secondary communications module 15 is configured to communicate with the monitoring devices 4 in a radio frequency protocol. However, any other suitable form of communications may be used.

A primary data storing means, in this embodiment of the invention a primary memory 17 is located in the mobile data collection device 5 for storing the monitored data and cross-referenced with the identity of the respective animals uploaded from the respective monitoring devices 4.

The microcontroller 8 of the mobile data collection device 5 may be programmed to operate the primary data communications modules 10 to transmit the data stored in the primary memory 17 to the central server 12 on completion of the uploading of the data from the monitoring devices 4 to the mobile data collection device 5. Alternatively, the microcontroller 8 may be programmed to operate the primary data communications module 10 of the mobile data collection device 5 to transmit the data stored in the primary memory 17 to the central server 12 on return of the drone 7 to a docking station 19 of the drone 7. When the drone 7 is docked in the docking station 19 the data indicative of the behaviour states and characteristics of the animals stored and cross-referenced in the primary memory 17 may be wirelessly uploaded to the central server 12 or through a USB port or other suitable hard wired communications means. Additionally, the microcontroller 8 may be programmed to operate the primary data communications module 10 to transmit data stored in the primary memory 17 to the central server 12 in response to an instruction signal received by the primary data communications module 10 from the central server 12 through the primary data communications module 10.

A location tracking module 20 is located in the mobile data collection device 5 for determining the location of the data collection device 5, and in turn for determining the location of each of the animals 2. The location tracking module 30, in this embodiment of the invention comprises a GPS tracking system. The location of each animal 2 is detected and recorded under the control of the microcontroller 8 and stored in the primary memory 17. The stored recorded locations of each of the animals 2 being monitored is transmitted to the central server 12 under the control of the microcontroller 8 of the mobile data collection device 5 to the central server 12 through the primary communications module 10 along with the data indicative of the behaviour, the states and the characteristics of the animals 2 stored in the primary memory 17 and cross-referenced therein with the identity of the corresponding animal. The data received from the mobile data collection device 5 by the central server 12 is stored in a suitable memory, for example, in the cloud, and is accessible by a farmer as it is required.

A power source, in this embodiment of the invention a battery 21 is located in the mobile data collection device 5 for powering the device 5, and in particular, for powering the microcontroller 8, the first secondary communications module 15, the primary data communications module 10 and the location tracking module 20. In this embodiment of the invention the battery 21 is a rechargeable battery. A solar panel, in this embodiment of the invention a photovoltaic solar panel 23 is mounted externally on the mobile data collection device 5 and is configured for charging the battery 21. However, it is envisaged that instead of the mobile data collection device 5 being powered by the battery 21 and the photovoltaic solar panel 23, the mobile data collection device 5 may be powered by the power source of the drone 7, or alternatively, the battery 21 may be rechargeable but would be charged when the drone 7 returns to and is engaged in the docking station 19.

Referring now to the monitoring devices 4, in this embodiment of the invention each monitoring device 4 is attached to the corresponding animal 2 by a neck band extending around the neck of the animal, and is weighted downwardly in order to depend from the neck of the animal. However, in other embodiments of the invention, it is envisaged that each monitoring device may be of the type which would be implanted in the corresponding animal, either just below the skin of the animal, or more deeply below the subcutaneous layer of the skin of the animal, and in other embodiments of the invention, it is envisaged that each monitoring device may be attached to one of the ears of the corresponding animal, or indeed, may be attached to the tail of the animal, to one of the legs of the animal, particularly, at a location towards the corresponding foot of the animal, or in any other suitable location in or on the animal.

Each monitoring device 4 comprises suitable sensors for monitoring the behaviour, the states and the characteristics of the corresponding animal to be monitored. For example, for monitoring for feeding, resting ruminating and the activity states of the animals, each monitoring device 4 comprises a two or three axis accelerometer 25. Such two or three axis accelerometers are discussed in the above referenced PCT specifications. Needless to say where the monitoring devices are to be provided for monitoring the temperature of the animals, the heart rate, blood pressure and the like of the animals, other suitable sensors 27 are located in each monitoring device 4.

Each monitoring device 4 comprises a secondary control means, in this embodiment of the invention a signal processor provided by a microprocessor 29 for controlling the operation of the monitoring device 4, and for reading signals from the accelerometer 25 and the sensors 27. The microprocessor 29 samples the signals from the accelerometer 25 and the sensors 27 of the corresponding monitoring device 4 at a sampling rate of 12.5 Hz, and buffers the sampled signals during consecutive predefined monitoring time periods. At the end of each predefined monitoring time period, the buffered data for that predefined monitoring time period is processed by the microprocessor 29 to determine the behaviour, states and/or characteristics of the corresponding animal 2 during that predefined monitoring time period, and the data indicative of the behaviour, states and/or characteristics of the corresponding animal during that predefined monitoring time period is stored and time stamped in a secondary storing means, namely, a secondary memory 30 located in the corresponding monitoring device 4. Alternatively, the raw buffered data sampled from the accelerometer 25 and the sensors 27 during each predefined monitoring time period may be stored and time stamped in the secondary memory 30 at the end of each predefined monitoring time period for further processing by the central server 12. In this embodiment of the invention each predefined monitoring time period is fifteen minutes, although it is envisaged that each predefined monitoring time period may range from five minutes and sixty minutes, depending on the behaviour, states and/or characteristics being determined. Additionally, the signals from the accelerometer 25 and the sensors 27 may be sampled at any other suitable sampling rate, and the sampling rate at which the signals from the accelerometer 25 are sampled may be different to the sampling rate at which the signals from the sensors 27 are sampled.

A second secondary communicating means, namely, a second secondary communications module 32 is located in each monitoring device 4 for communicating data stored in the secondary memory 30 indicative of the behaviour, states and characteristics of the corresponding animal for reception by the mobile data collection device 5. In this embodiment of the invention the second secondary communications module 32 is configured to communicate with the first secondary communications module 15 of the mobile data collection device 5 in the radio frequency protocol. An identifying means, namely, an identifying code, identifying each monitoring device 4, and in turn the corresponding animal, is stored in the secondary memory 30 of the corresponding monitoring device 4. On transmission of the data stored in the secondary memory 30 of the corresponding monitoring device 4 by the second secondary communications module 32 to the mobile data collection device 5, the data being transmitted from the secondary memory 30 is cross-referenced with the identifying code of the monitoring device 4, which in turn identifies the corresponding animal 2. Each transmission of the time stamped data cross-referenced with the identity of the animal from each monitoring device 4 is initiated by a signal from the mobile data collection device.

The transmission range between the first and second secondary communications modules 15 and 32 in this embodiment of the invention is approximately seven metres. This has the advantage that since communication can only be carried out between the mobile data collection device 5 and each monitoring device 4 when the mobile data collection device 5 is within seven metres of the monitoring device 4, an approximate location of the animal corresponding to the monitoring device 4 with which communication is being carried out can be readily determined, since the microcontroller 8 of the mobile data collection device 5 can determine its location from the location tracking module 20, and thus, the location of the animal 2, the monitoring device 4 of which is in communication with the mobile data collection device 5 is therefore within a radius of 7 metres of the location of the mobile data collection device 5 determined from the location tracking module 20 thereof. Data indicative of the location of each animal 2 time stamped and cross-referenced with the identity of the animal 2 is stored in the primary memory 17 of the mobile data collection device 5.

A power source in this embodiment of the invention a long life on-board battery 34 is located in each monitoring device 4 for powering the monitoring device 4, and in particular, the microprocessor 29, the accelerometer 25, the sensors 27 and the second secondary communications module 32. The battery 34 may be a rechargeable or a non-rechargeable battery, however, in this embodiment of the invention the battery 34 is a rechargeable battery.

In use, with the batteries 34 of the monitoring devices 4 fully charged and the microprocessors 29 of each monitoring device 4 programmed to read the signals from the accelerometer 25 and the sensors 27, the monitoring devices 4 are attached to the animals 2. The mobile data collection device 5 is mounted on the drone 7, and the drone 7 is programmed to fly past the animals 2 of the herd at the predefined data collection time intervals.

At the end of each predefined monitoring time period, the microprocessor 29 of each monitoring device 4 processes the buffered data sampled from the accelerometer 25 and the sensors 27 during that predefined monitoring time period to determine the behaviour, the states and/or the characteristics of the corresponding animal. The processed data indicative of the behaviour, states and/or characteristics of the corresponding animal 2 is time stamped and stored in the secondary memory 30 for subsequent transmission to the mobile data collection device 5. This process is repeated during and at the end of each predefined monitoring time period by the microprocessor 29 of each of the monitoring devices 4.

At the end of each predefined data collection time interval, the drone 7 with the mobile data collection device 5 secured thereto takes-off from the docking station 19 to commence a fly pass of the animals 2. On reaching the animals 2, the mobile data collection device 5 commences to sequentially communicate with the monitoring devices 4 of the animals 2 as the mobile data collection device 5 comes within 7 metres of the respective monitoring devices 4. Data communication between the mobile data collection device 5 and each monitoring device 4 is initiated by an initiating signal transmitted under the control of the microcontroller 8 of the mobile data collection device 5 through the first secondary communications module 15 to initiate the second secondary communications module 32 of the corresponding monitoring device 4. The time stamped data indicative of the behaviour, states and/or characteristics of the corresponding animal 2 is transmitted cross-referenced with the identifying code of the monitoring device 4 by the second secondary communications module 32 to the first secondary communications module 15 of the mobile data collection device 5. The time stamped data indicative of the behaviour, states and/or characteristics of the corresponding animal 2 cross-referenced with the identity of the corresponding animal 2 is read through the first secondary communications module 15 by the microcontroller 8 of the mobile data collection device 5, and is stored in the primary memory 17 of the mobile data collection device 5. As the data stored in the secondary memory 30 of each monitoring device 4 is being uploaded via the first and second secondary communications modules 15 and 32, the microcontroller 8 reads the current position of the mobile data collection device 5 from the location tracking module 20 in order to determine the location of the animal 2 corresponding to the monitoring device 4 from which the data is being uploaded to the mobile data collection device 5. The data indicative of the current location of the animal corresponding to the monitoring device 4 from which data is being uploaded to the mobile data collection device 5 is time stamped and cross-referenced with the identity of the corresponding animal and stored in the primary memory 17 of the mobile data collection device 5 along with the data uploaded from the secondary memory 30 indicative of the behaviour, states and/or characteristics of that animal 2.

This process is continued by the mobile data collection device 5 until the data indicative of the behaviour states and/or characteristics of the respective animals 5 have been uploaded from all the monitoring devices 4 to the mobile data collection device 5.

On completion of uploading of the data indicative of the behaviour, states and/or characteristics of the animals 2 from the secondary memories 30 of the respective monitoring devices 4 to the primary memory 17 of the mobile data collection device 5, the data indicative of the behaviour, states and/or characteristics and locations of the animals 2 cross-referenced with the identities of the animals 2 stored in the primary memory 17 may be transmitted directly by the primary communications module 10 to the central server 12 via the GSM network 14. Alternatively, the data stored in the primary memory 17 may be uploaded from the primary memory 17 of the mobile data collection device 5 to the central server 12 when the drone 7 has returned to and is docked in the docking station 19.

Additionally, on receiving a request from the central server 12 to transmit data to the central server 12, the microcontroller 8 of the mobile data collection device 5 transmits the relevant data from the primary memory 17 to the central server 12 via the GSM network 14.

Referring now to FIG. 4, there is illustrated a data collection system according to another embodiment of the invention indicated generally by the reference numeral 35 for collecting data indicative of one or more of the behaviour, states and/or characteristics of a plurality of animals 2, and for transmitting the collected data to a central server 12. In this embodiment of the invention the data collection system 35 is substantially similar to the data collection system 1 described with reference to FIGS. 1 to 3, and similar components are identified by the same reference numerals. The only significant difference between the data collection system 35 and the data collection system 1 is that instead of the mobile data collection device 5 being mounted on a drone, the mobile data collection device 5 is mounted on one of the animals 2, namely, the animal 2a. In this embodiment of the invention the microcontroller 8 of the mobile data collection device 5 is programmed so that when the animal 2a and in turn the mobile data collection device 5 comes within transmission distance of the monitoring devices 4 of each of the animals 2, the mobile data collection device 5 transmits a data transmission initiation signal which is received by the adjacent monitoring device 4 to initiate communications between the monitoring device 4 and the mobile data collection device 5 for transmitting the time stamp data indicative of the behaviour, states and/or characteristics of the corresponding animal 2 stored in the secondary memory 30 of that monitoring device 4 cross-referenced with the identity of the animal to the mobile data collection device 5 for storing in the primary memory 17 for subsequent transmission to the central server 12. In this embodiment of the invention in order to ensure that the mobile data collection device 5 communicates with the monitoring devices 4 of all the animals, the transmission range between the first and second secondary communications modules 15 and 32 is greater than seven metres and may be as much as 100 metres and in some embodiments of the invention the transmission range between the first and second secondary communications modules 15 and 32 may be as much as one kilometre. Thus, depending on the transmission range between the first and second secondary communications modules 15 and 32, it may or may not be possible to determine the location of the animals 2 using the location tracking module 20 of the mobile data collection device 5.

The microcontroller 8 of the mobile data collection device 5 is programmed to transmit the collected data stored in the primary memory 17 to the central computer 12 through the communications network 14 at predefined transmission time intervals. The predefined transmission time intervals may be of any desired duration, and may be of duration anywhere from 1 hour to 24 hours, depending on the frequency with which the data indicative of the behaviour, states and/or characteristics of the animals 2 is to be updated in the central server 12. However, in this embodiment of the invention each predefined transmission time interval is of duration of approximately 6 hours.

In this embodiment of the invention the animal 2a on which the mobile data collection device 5 is located is also provided with a monitoring device 4, and at predefined data collection time intervals the microcontroller 8 is programmed to initiate transmission between the mobile data collection device 5 and the monitoring device 4 of the animal 2a to upload the stored data indicative of the behaviour, states and/or characteristics of the animal 2a from the monitoring device 4 to the mobile data collection device 5 for storing in the primary memory 17 of the mobile data collection device 5 for subsequent transmission to the central server 12.

In this embodiment of the invention the mobile data collection device 5 is mounted on the neck of the animal 2a adjacent the top of the neck of the animal 2a in order to facilitate data transmission between the monitoring devices 4 of the other animals 5 and the mobile data collection device 5. The mobile data collection device 5 is secured adjacent the top of the neck of the animal 2a by a neck strap, and typically, would be mounted on the neck of the animal by the same neck strap as the monitoring device 4 of that animal 2a.

Otherwise, the data collection system 35 and its use is similar to the data collection device 1 described with reference to FIGS. 1 to 3.

Referring now to FIGS. 5 and 6 there is illustrated a data collection system according to another embodiment of the invention indicated generally by the reference numeral 40 for collecting data and processing the data to determine the behaviour and states of a plurality of animals 41. The animals 41 in this case are also suckler cows and are also illustrated in block representation. The data collection system 40 comprises a mobile data collection device 42 which is configured for collecting data indicative of the behaviour and states of the animals 41, and is also configured for processing the data to determine the behaviour and states of the animals 41. The data collection system 40 is configured for mounting on a prime mover, which in this embodiment of the invention may also be a drone 43, similar to the drone 7 of the data collection system 1. A docking station 44 to which the drone 43 docks is provided. Alternatively, the prime mover may be a land vehicle, which typically would be an autonomous land vehicle. If the prime mover is an autonomous land vehicle, the land vehicle would incorporate autonomous driving technology. However, in this embodiment of the invention monitoring devices similar to the monitoring devices 4 of the data collection system 1 are not required.

The mobile data collection device 42 comprises a data acquisition means which in this embodiment of the invention comprises an imaging means, namely, a video camera 45 provided by a charge coupled device. The video camera 45 is configured for capturing images and video clips of each animal, as the mobile data collection device 42 passes the animal. In this embodiment of the invention the mobile data collection device 42 is configured to determine the behaviour and states as well as the identity of each animal from captured images and video clips of that animal captured by the video camera 45.

An image recognition means, namely, an image recognition module 47 is located in the mobile data collection device 42 and stores reference images of the respective animals 41 for comparison with captured images of the animals 41 captured by the video camera 45 for determining the identity of each animal.

A data analysing means, namely, a data analysis module 48 is located in the mobile data collection device 42 for analysing the captured images and the captured video clips of the respective animals 41 for identifying the behaviour and state of the animals 41, as will be described below.

A location tracking module 49 is located in the mobile data collection device 42 for determining the location of the mobile data collection device 42.

A control means, in this embodiment of the invention a signal processor, which is provided by a microprocessor 50 controls the operation of the mobile data collection device 42, and reads signals from the video camera 45, the image recognition module 47, the data analysing module 48 and the location tracking device 49. A primary storing means in this embodiment of the invention comprising a first primary memory 52 and a second primary memory 54 are located in the mobile data collection device 42. Raw data read by the microprocessor 50 from the video camera 45 and the location tracking module 49 is time stamped by the microprocessor 50 and is written by the microprocessor 50 to the first primary memory 52 and cross-referenced with the identities of the respective animals. The microprocessor 50 is programmed in conjunction with the data analysing module 48 to process the raw data stored in the primary memory 52 for each animal in order to determine the behaviour and states of the animal from that data. The processed data indicative of the behaviour and states of the respective animals determined by the microprocessor 50 and time stamped with the time of the corresponding raw data is stored in the second primary memory 54 cross-referenced with the identities of the respective animals 41. The microprocessor 50 reads the current location of the mobile data device 42 from the location tracking module 49 in order to determine the location of each animal 41. Data indicative of the location of each animal time stamped is stored in the second primary memory 54 cross-referenced with the identity of the corresponding animal 41.

A primary communicating means, in this embodiment of the invention a primary two-way (transmitter/receiver) wireless communications module 55 is located in the mobile data collection device 42 for wirelessly communicating through a wireless communications network 56, in this embodiment of the invention a GSM network 56, the time stamped data indicative of the behaviour, states and location of the respective animals 41 from the second primary memory 54 to a central server 58, which in this embodiment of the invention is located adjacent the docking station 44 of the drone 43.

A power source, in this embodiment of the invention a rechargeable battery 60 powers the mobile data collection device 42, including, the video camera 45, the image recognition module 47, the data analysing module 48, the location tracking module 49, the microprocessor 50 and the primary communications module 55. In this embodiment of the invention the battery 60 is a rechargeable battery. A solar panel, in this embodiment of the invention a photovoltaic panel 62 is mounted on the mobile data collection device 42 for powering the mobile data collection device 42 and for charging the battery 50 thereof.

In use, with the mobile data collection device 42 secured to the drone 43 and with the drone 43 programmed to fly past the animals 41 at the predefined data collection time intervals, the system 40 and the mobile data collection device 42 is ready for use. At the end of each predefined data collection time interval, the drone 43 takes off from its docking station 44 and commences a pass of the animals 41.

As the drone 43 reaches the animals, the drone 43 hovers adjacent the respective animals 41 in order to allow time for the video camera 45 to sequentially capture images and video clips of the respective animals 41 for subsequent analysis. The raw data signals from the video camera 45 are read by the microprocessor 50, are time stamped and stored in the first primary memory 52. Data indicative of the images of the respective animals is written to the image recognition module 47 by the microprocessor 50, and the image recognition module 47 identifies the animals from the data indicative of the captured images thereof by comparing the respective captured images with the reference images of the animals. The identity of the respective animals 41 is then cross-referenced with the captured image data by the microprocessor 50 and the image data is stored in the first primary memory 52 cross-referenced with the identity of the respective animals.

The data of the video clips is written by the microprocessor 50 to the data analysing module 48, which analyses the video clip data of the respective animals 41 to determine the behaviour and states of the respective animals 41. The activity level of each animal is determined by the data analysing module 48 from the amount of activity of that animal identified from the video clip of that animal. On completing the analysis of the video clips of the respective animals 41, the data analysing module 48 determines whether the level of activity of each animal is at a high level, a medium level or a low level.

Additionally, the data analysing module also determines if each animal is eating from the video clip of that animal by monitoring the position of the head of the animal relative to the body of the animal, and the angle of the neck of the animal if the animal is standing with the head down and the neck of the animal inclining downwardly, that would be an indication of an animal eating.

Ruminating of an animal is determined by the data analysing module 48, from the position of the head and neck of the animal and the mouth movements of the animal. If the mouth of the animal is moving and the head of the animal is up with the neck extending substantially horizontally, that would be an indication of a ruminating animal.

The data analysing module 48 also determines from the video clip of each animal if an animal is lame, is spending an excessive amount of time lying down.

Additionally, the data analysis module 48 is configured to determine if any of the animals 41 are mounting another animal 41, which if the animal 41 being mounted remains still would be an indication that the standing animal 41 could be in heat. If a mounted animal 41 were determined to remain standing for an appropriate time during mounting corresponding to the length of time an animal in heat would allow herself to be mounted, that standing animal 41 would be determined as being in heat, and time stamped data indicative of the state of the animal 41 as being in heat would be stored and cross-referenced with the identity of the animal 41 in the second primary memory 54.

These states of the animal are read from the data analysing module 48 by the microprocessor 50 which time stamps the data based on the time stamp of the corresponding raw data on which the analysis has been carried out, and the time stamped data indicative of the behaviour and states of the animals 41 cross-referenced with the identity of the respective animals 41 is written to and stored in the second primary memory 54.

Additionally, the microprocessor 50 reads the location of the mobile data collection device 43 from the location tracking module 49 as a video clip of each animal is being captured by the video camera 45. Time stamped data indicative of the location of the animal cross-references with the identity of the animal is written to and stored in the second primary memory 54 by the microprocessor 50. The data analysing module 48 is programmed to determine from the size of the image of the animal in the video clip a rough indication of the distance of the animal from the mobile data collection device 42. Thus, by knowing the location of the mobile data collection device 42 from the data read from the location tracking module 49, the location of the animal can be determined to be within a radius of the distance of the animal from the mobile data collection device 42 determined by the data analysing module 48.

On determining the behaviour, states and the location of the respective animals 41, the microprocessor 50 activates the primary communications module 55 to transmit the data indicative of the behaviour and states of the animals as well as the locations of the animals to the central server 58. Alternatively, the data indicative of the behaviour, states and locations of the respective animals may be communicated to the central server 58 on docking of the drone 43 in the docking station 44. In this embodiment of the invention since the docking station 44 is located adjacent the central server 58, the data stored in the second primary memory 54 may be uploaded to the central server through a USB connection in the docking station 44.

Referring now to FIGS. 7 and 8 there is illustrated a mobile data collection system according to another embodiment of the invention indicated generally by the reference numeral 70 for monitoring at least one of the behaviour, at least one state and at least one characteristic of a plurality of animals 71 and for collecting data indicative of the at least one of the behaviour, the at least one state and the at least one characteristic thereof. The animals 71 are illustrated in block representation only. The data collection system 70 is substantially similar to the data collection system 40, and similar components are identified by the same reference numerals. The data collection system 70 comprises a mobile data collection device 42 similar to the mobile data collection device 42 of the data collection system 40. Similarly, as in the case of the data collection system 40, the animals 71 are not provided with monitoring devices similar to the monitoring devices 4 of the mobile data collection system 1.

However, in this embodiment of the invention the mobile data collection device 42 instead of being mounted on a prime mover provided by a drone, the mobile data collection device 42 is mounted directly on to one of the animals 71, namely, the animal 71a. Therefore, as that animal 71a with the mobile data collection device 42 mounted thereon moves around relative to the other animals 71, the video camera 45 is continuously operated for capturing images and video clips of the other respective animals 71 as the other respective animals 71 come within range of the video camera 45. As an image and a video clip of each animal 71 is being captured by the video camera 45, the raw data is read from the video camera 45 by the microprocessor 50 and is time stamped by the microprocessor 50 and stored in the first primary memory 52 for subsequent processing as already described with reference to the mobile data collection device 42 of the data collection system 40.

In this embodiment of the invention the mobile data collection device 42 is mounted on the neck of the animal 71a adjacent the top of the neck, and adjacent the head of the animal. The video camera 45 is located adjacent the top of the mobile data collection device 42 in order to maximise the field of view of the video camera 45.

The identities of the respective animals 71 and the behaviour and states of the respective animals 71 are determined by the image recognition module 47 and the data analysing module 48 in conjunction with the microprocessor 50 as already described. The location of the respective animals 71 is also determined by the microprocessor 50 from the the data read from the location tracking module 49. The data indicative of the behaviour and states of the respective animals 71 and their respective locations cross-referenced with their respective identities, all of which is time stamped by the microprocessor 50 is then stored in the second primary memory 54 for subsequent transmission to the central server 58.

In this embodiment of the invention the microprocessor 50 is programmed to activate the primary communications module 55 at predefined transmission time intervals to upload the time stamped data stored in the second primary memory 54 indicative of the behaviour, states and locations of the respective animals 71 at the respective times to the central server 58 via the GSM network 56. In this embodiment of the invention the predefined transmission time intervals are six hourly intervals. Although, it will be appreciated that the predefined transmission time intervals may be of any suitable time duration. In general, the time duration of each predefined transmission time interval will be a matter of optimising between the frequency with which it is desired that the data be transmitted from the mobile data collection device 42 to the central server 55 and battery life of the battery 60 of the mobile data collection device 42, which in turn will be dependent upon the efficiency of the solar panel and the available sunlight. However, typically, the predefined transmission time intervals will be of time duration lying in the range of 6 hours to 24 hours, but may be longer than 24 hours or shorter than 6 hours.

The microprocessor 50 is programmed that in the event of an emergency being detected in connection with any of the animals 71, the microprocessor 50 activates the primary communications module 55 to upload data indicative of the behaviour and/or state of the animal in which the emergency has been detected to the central server 58, together with the relevant time stamped data indicative of the emergency cross-referenced with the identity of the animal.

Additionally, the microprocessor 50 is programmed in response to a command signal received through the primary communications module 55 from the central server 58 to operate the primary communications module 55 to transmit to the central server 58 the data stored in the second primary memory 54 indicative of the behaviour and/or states of the animals cross-referenced with the identities of the respective animals 71.

Otherwise, the data collection system 70 is similar to the data collection system 40.

In the embodiments of the invention described with reference to FIGS. 5 to 8 it is not possible to monitor some characteristics of the animal, for example, the temperature, heart rate and blood pressure of the animal, although where it is desired to monitor the temperature of the animals the mobile data collection device may be provided with an infra-red temperature sensor or other suitable temperature sensor for monitoring the temperature of an animal within a range of, for example, up to ten metres.

While the mobile data collection device 42 of the data collection systems 40 and 70 have been described as comprising an image recognition module 47 for identifying each of the animals, it is envisaged that in certain cases, the image recognition module may be omitted. In which case each animal may be provided with an identification means, for example, an identification tag, which could comprise either a visual identification, for example, a visually readable code of the animal, or an electronic identification, for example, an electronically readable code of the animal. In the event of each identification tag comprising electronic identification of the corresponding animal, it is envisaged that each data identification tag would be provided with an RFID module for passively uploading the identification data of the corresponding animal from the electronic tag to the mobile data collection device 42. Alternatively, in the event of each tag comprising a visual identification of the corresponding animal, for example, an alpha numeric code, a barcode, a OR code, or any other such code, it is envisaged that the data collection device 42 would be provided with a suitable code reader for reading the code and in turn identifying the animal from the read code.

The mobile data collection device 42 of the data collection system 70 is particularly suitable for detecting an animal in heat. For example, if the animal with the mobile data collection device located thereon was determined by the mobile data collection device as being in a mounting state, mounting another one of the animals, and if that animal with the mobile data collection device located thereon was allowed by the other animal to remain in the mounting state for an appropriate period of time corresponding to the length of time an animal in heat would allow herself to be mounted, the mobile data collection device 42 would be programmed to determine that the animal being mounted is in heat. The mobile data collection device 42 would read or determine the identity of the animal which is in closest proximity to it, namely, the animal being mounted, in order to identify that animal, and the identity of the animal being mounted, together with the determination of the heat state of that animal being mounted as being in heat would be stored, time stamped and cross-referenced with her identity and stored in the second primary memory 54 of the mobile data collection device 42 for subsequent transmission to the central server 58 as already described.

Conversely, if the animal with the mobile data collection device 42 mounted thereon was detected remaining in a standing state while being mounted by another animal for a time period corresponding to the length of time an animal in heat would remain standing while being mounted, the mobile data collection device 42 would determine the animal with the mobile data collection device located thereon was in heat, and store the heat status of the animal, time stamped and cross-referenced with her identity in the second primary memory 54 of the mobile data collection device 42 for subsequent transmission to the central server.

While the mobile data collection devices have been described as comprising either a microcontroller or a microprocessor, it is envisaged that in certain cases, the mobile data collection devices may be provided with either a microprocessor or a microcontroller, instead of a microcontroller or a microprocessor, as the case may be. Additionally, in either case, the microprocessor or microcontroller may be programmed to carry out either or both the functions of the image recognition module and the data analysis module, and the microprocessor or the microcontroller may also be configured to incorporate the location tracking module.

While the data collection devices 5 and 42 of the systems 1 and 40 have been described for mounting on a drone, and have been described as being mounted on a drone 17, it is envisaged that the data collection devices may be configured for mounting on any prime mover capable of urging the data collection devices to and past the animals. For example, the data collection devices may be configured for mounting on a land vehicle, which may be autonomous or otherwise, and when configured for mounting on a land vehicle, the land vehicle may, for example, be a quad-bike which could be configured as an autonomous quad-bike. It is also envisaged that the data collection devices may be configured for mounting on a tractor be it an autonomous tractor or an non-autonomous tractor, which would be driven by a farmer.

While the mobile data collection device 5 of the system 1 has been described as comprising a first secondary communications module for communicating with the monitoring devices 4, any other suitable communicating means may be provided for communicating with the monitoring devices and for uploading data from the monitoring devices to the mobile data collection device 5.

While the mobile data collection devices 42 of the data collection systems 40 and 70 have been described as comprising a data analysis module 48 for analysing the data captured by the video camera, it is envisaged that in some embodiments of the invention the data analysis module 48 would be omitted, and in which case, the microprocessor 50 or a microcontroller would be programmed to operate the primary communications module 55 for transmitting the raw data to the central server, for subsequent analysis by the central server.

It is also envisaged that while the rechargeable battery of the mobile data collection devices 5 and 42 have been described as being charged by a photovoltaic solar panel, the rechargeable batteries of the mobile data collection devices 5 and 42 may be charged by any other suitable available power source, be it a renewable power source or otherwise, for example, an energy converter, such as a wind energy converter, a motion energy converter or any other such energy converter may be provided for converting wind, motion or other energy to electrical energy for charging the battery of the mobile data collection devices.

While the first and second secondary communications modules of the mobile data collection device 5 and the monitoring devices 4 of the embodiments of the invention described with reference to FIGS. 1 to 4 have been described as being configured to communicate in a radio frequency protocol, the first and second secondary communications modules may be configured to wirelessly communicate in any other suitable protocol or in any other suitable manner. It is envisaged that in some embodiments of the invention that the second secondary communications modules of the monitoring devices may be configured to be powered by the first secondary communications modules of the mobile data collection device during transmission of the data indicative of the behaviour, states and/or characteristics of the animals from the second secondary communications modules to the first secondary communications module. Such powering of the second secondary communications modules by the first secondary communications module could be carried out by electromagnetic induction provided by electromagnetic induction signals transmitted from the first secondary communications module to the second secondary communications module of the monitoring device which is in communication with the mobile data collection device. Additionally, it is envisaged that the communications between the mobile data communications module and the monitoring devices may be carried out possibly by providing the mobile data communications device with a first RFID communications module and the monitoring devices with passive second RFID communications modules whereby the first RFID communications module of the mobile data collection device would power the second RFID communications modules of the respective monitoring devices during communications therewith.

While the mobile data collection devices have been described as comprising a data acquisition means in the form of a video camera, it will be readily apparent to those skilled in the art that the data acquisition means may comprise any suitable imaging means. Indeed, where the mobile data collection device is provided with a data acquisition means comprising a video camera, it is envisaged that the video camera may be provided with a fish eye lens in order to enhance the field of view of the video camera. It is also envisaged that the mobile data collection device may be provided with more than one video camera, for example, two, three, or four video cameras, each of which would be provided with a fish eye lens, and the video cameras would be configured with the respective fish eye lenses located spaced apart around the mobile data collection devices to provides a 360° viewing angle around an axis defined by the mobile data collection device extending substantially upwardly and perpendicularly from the neck of the animal on which the mobile data collection device is located.

While the mobile data collection devices of the data collection systems according to the invention have been described as being specific mobile data collection devices, it is envisaged that in certain cases, the mobile data collection device of each data collection system may be provided in the form of a smart mobile device, for example, a smart mobile phone, which would be configured to carry out the functions of the method according to the invention for collecting and transmitting the data indicative of the behaviour, states and/or characteristics of the animals of a plurality of animals. In which case, the mobile phone may be configured to be mounted on one of the animals, or may be configured to be mounted on a prime mover, for example, a drone, a land vehicle, such as, for example, a quad bike, a tractor or the like.

It will be appreciated that in the embodiments of the invention in which the mobile data collection device comprises an imaging means, for example, a video camera or video cameras, any suitable characteristics of the animals may be selected for identifying the animals. For example, in the case of piebald animals, the piebald pattern of the animals, which in general is distinctive of each animal may be used for identifying the respective animals. Alternatively, the animals may be identified by characteristics of the eye or the like of the animals, or by any other characteristics of the animal. Additionally, the animals may be provided with a visually readable code or pattern an image of which would be captured by the imaging means, and processed for identifying the animal. Such a visually readable code, may, for example, be a numeric code, an alpha numeric code, a bar code, a OR code, or any other suitable visually readable distinctive code or pattern.

1-141. (canceled)

142. A method for monitoring at least one of the behaviour, at least one state or at least one characteristic of each of a plurality of animals, the method comprising acquiring data indicative of the at least one of the behaviour, the at least one state or the at least one characteristic of each of the animals by a mobile data collection device, storing the acquired data in a primary storing means of the mobile data collection device, communicating the acquired data stored in the primary storing means of the mobile data collection device to a central server, and mounting the mobile data collection device on one of a prime mover and one of the animals.

143. A method as claimed in claim 142 in which the data indicative of the at least one of the behaviour, the at least one state or the at least one characteristic of the animals is acquired by the mobile data collection device by a data acquisition means, and preferably, the data acquisition means comprises an imaging means, and advantageously, the data acquisition means of the mobile data collection device comprises an imaging device, and preferably, the data acquisition means comprises one of a camera and a video camera, and advantageously, the data acquisition means comprises a charge coupled imaging device.

144. A method as claimed in claim 142 in which data indicative of the identity of each of the animals is acquired by the mobile data collection device, and preferably, the data indicative of the identity of each animal is stored and cross-referenced with the determined one of the behaviour, the at least one state or the at least one characteristic of the animal in the primary storing means of the mobile data collection device, and advantageously, the data indicative of the identity of each of the animals is determined from at least one image of the animal captured by the imaging means, and preferably, the identity of each animal is determined from a plurality of images sequentially captured by the imaging means, and advantageously, the identity of each animal is determined by an image recognition means configured for identifying the respective animals from respective images thereof captured by the imaging means, and preferably, the image recognition means is located in the mobile data collection device, and advantageously, the at least one of the behaviour, the at least one state or the at least one characteristic of each animal is determined from one or more images of the animal captured by the imaging means, and preferably, the at least one of the behaviour, the at least one state or the at least one characteristic of each animal is determined from a series of images of the animal sequentially captured by the imaging means, and advantageously, the images captured by the imaging means are analysed for determining the at least one of the behaviour, the at least one state or the at least one characteristic of the animal, and preferably, a series of the images captured by the imaging means of the mobile data collection device are analysed for determining the at least one of the behaviour, the at least one state or the at least one characteristic of a corresponding one of the animals.

145. A method as claimed in claim 142 in which each animal is provided with an identifying means comprising data indicative of the identity of the corresponding animal, and preferably, the identifying means of each animal comprises the data indicative of the identity of the animal in the form of an electronically readable identifying code, and advantageously, the identifying means of each animal comprises a vision readable identity code, and preferably, the identifying code is read from the identifying means by the data acquisition means of the mobile data collection device, and advantageously, the identifying means is located one of externally on and internally in the animal.

146. A method as claimed in claim 142 in which the mobile data collection device comprises a location tracking means, and preferably, the location tracking means cooperates with a GPS network for identifying the location of the mobile data collection device, and advantageously, the location of each animal is determined by the location tracking means of the mobile data collection device, and preferably, the mobile data collection device communicates data indicative of the location of each animal cross-referenced with the data indicative of the identity of the animal to the central server, and advantageously, the mobile data collection device communicates the data indicative of the one of the behaviour, the at least one state or the at least one characteristic of each animal cross-referenced with the identity of the animal to the central server wirelessly, and preferably, the mobile data collection device communicates with the central server via a wireless communications network, and advantageously, the mobile data collection device communicates with the central server via a telecommunications network, and preferably, the mobile data collection device communicates with the central server via a GMS communications network, and advantageously, the data indicative of the at least one of the behaviour, the at least one state or the at least one characteristic of each animal is communicated to the central server by the mobile data collection device just after it has been stored in the primary storing means of the mobile data collection device.

147. A method as claimed in claim 142 in which the mobile data collection device is mounted on one of the animals, and preferably, the data indicative of the at least one of the behaviour, the at least one state or the at least one characteristic of each animal is communicated to the central server by the mobile data collection device at predefined transmission time intervals, and advantageously, each predefined transmission time interval is of time duration in the range of 3 hours to 24 hours, and preferably, each predefined transmission time interval is of time duration in the range of 4 hours to 12 hours, and advantageously, each predefined transmission time interval is of time duration of approximately 6 hours.

148. A method as claimed in claim 142 in which the mobile data collection device is mounted on the prime mover and is urged past the animals by the prime mover at predefined data collection time intervals, and preferably, the time duration of each predefined data collection time interval lies in the range of 3 hours to 24 hours, and advantageously, the time duration of each predefined data collection time interval lies in the range of 4 hours to 12 hours, and preferably, the time duration of each predefined data collection time interval is approximately 6 hours, and advantageously, the mobile data collection device is mounted on the prime mover, so that the mobile data collection device is urged past the respective animals within a distance of up to 10 metres of each animal, and preferably, the mobile data collection device is mounted on the prime mover, so that the mobile data collection device is urged past the respective animals within a distance of up to 7 metres of each animal, and advantageously, the mobile data collection device is mounted on the prime mover, so that the mobile data collection device is urged past the respective animals within a distance of approximately 3 metres of each animal, and preferably, the mobile data collection device is mounted on the prime mover in the form of one of a drone configured for flying, a tractor and a quad-bike, and advantageously, the data acquired by the mobile data collection device is communicated to the central server on completion of each pass of the animals by the mobile data collection device, and preferably, the data indicative of the at least one of the behaviour, the at least one state or the at least one characteristic of each animal acquired by the mobile data collection device is communicated by the mobile data collection device to the central server by a hard wire connection.

149. A method as claimed in claim 142 in which each animal is provided with a monitoring device configured for monitoring the at least one of the behaviour, the at least one state or the at least one characteristic of the corresponding animal, and preferably, each monitoring device is configured for monitoring the at least one of the behaviour, the at least one state or the at least one characteristic of the corresponding animal during predefined monitoring time periods, and at the end of each predefined monitoring time period data captured during that predefined monitoring time period indicative of the at least one of the behaviour, the at least one state or the at least one characteristic is stored, and advantageously, the time duration of each predefined monitoring time period lies in the range of 5 minutes to 60 minutes, and preferably, the time duration of each predefined monitoring time period lies in the range of 10 minutes to 45 minutes, and advantageously, the time duration of each predefined monitoring time period is approximately 15 minutes, and preferably, the predefined monitoring time period run consecutively one after the other, and advantageously, the identifying means comprising the identity of each animal is located in the monitoring device of the corresponding animal.

150. A method as claimed in claim 149 in which each monitoring device comprises a secondary storing means configured for storing data indicative of the at least one of the behaviour, the at least one state or the at least one characteristic of the corresponding animal cross referenced with the identity of the animal, and preferably, the data stored in the secondary storing means of each monitoring device is acquired from the monitoring devices by the data acquisition means of the mobile data collection device, and advantageously, the data stored in the secondary storing means of each monitoring device is acquired wirelessly by the data acquisition means of the mobile data collection device, and preferably, the data stored in the secondary storing means of each monitoring device is acquired wirelessly by the data acquisition means of the mobile data collection device by radio frequency protocol, and advantageously, the data stored in the secondary storing means of each monitoring device is acquired wirelessly by the data acquisition means of the mobile data collection device by passive communication, under which the monitoring device is powered wirelessly by the mobile data collection device, and preferably, the data stored in the secondary storing means of each monitoring device is acquired by the data acquisition means of the mobile data collection device by RFID communications, and advantageously, the data stored in the secondary storing means of each monitoring device is acquired by the data acquisition means of the mobile data collection device by passive RFID communications, and preferably, each monitoring device is configured for mounting one of externally on and internally in the corresponding animal, and advantageously, each monitoring device is activated by a signal from the mobile data collection device to initiate acquisition of data from the storing means in the corresponding mobile device by the mobile data collection device.

151. A method as claimed in claim 142 in which the at least one of the behaviour, the at least one state or the at least one characteristic of each animal relates to one or more of resting, feeding, ruminating and the level of activity of the animal, and preferably, the level of activity monitored of each animal is categorised as being one of a highly active state or a low active state, and advantageously, the level of activity monitored of each animal is categorised as being one of a medium active state, the medium active state being of activity less than the highly active state, and activity greater than the low active state, and preferably, the at least one characteristic of each animal which is monitored comprises one of body temperature, blood pressure and heart rate, and advantageously, the data indicative of the at least one of the behaviour, the at least one state or the at least one characteristic of each animal is communicated to the central server by the mobile data collection device in response to receipt of a signal from the central server, and preferably, the data indicative of the one of the behaviour, the at least one state or the at least one characteristic of one or more of the animals is communicated to the central server by the mobile data collection device in response to the data indicative of the one of the behaviour, the at least one state or the at least one characteristic being indicative of an emergency state being detected in that one or more of the animals, and advantageously, the animals are remotely located.

152. A data collection system comprising a mobile data collection device configured for carrying out the method as claimed in claim 142 for monitoring at least one of the behaviour, at least one state or at least one characteristic of each of a plurality of animals.

153. A data collection system for collecting data relating to at least one of the behaviour, at least one state or at least one characteristic of a plurality of animals, the system comprising a mobile data collection device, the mobile data collection device comprising a data acquisition means for acquiring data indicative of the at least one of the behaviour, the at least one state, or the at least one characteristic of each animal, a primary storing means for storing the acquired data, and a primary communicating means for communicating the stored data in the primary storing means to a central server, the data collection device being configured for mounting on one of a prime mover and one of the animals.

154. A data collection system as claimed in claim 153 in which the data acquisition means comprises an imaging means, and preferably, the data acquisition means comprises an imaging device, and advantageously, the data acquisition means comprises one of a camera and a video camera, and preferably, the data acquisition means comprises a charged coupled imaging device, and advantageously, the mobile data collection device comprises an image recognition means configured for identifying each animal from at least one image of the animal captured by the imaging means, and preferably, the image recognition means is configured for identifying each animal from a series of images of the animal captured by the imaging means, and advantageously, the image recognition means is configured for identifying each animal from a series of images of the animal sequentially captured by the imaging means, and preferably, the mobile data collection device comprises an analysing means for analysing at least one image of each animal captured by the imaging means for determining the at least one of the behaviour, the at least one state or the at least one characteristic of the animal, and preferably, the analysing means is configured for analysing a series of images of each animal captured by the imaging means for determining the at least one of the behaviour, the at least one state or the at least one characteristic of the animal, and advantageously, the analysing means is configured for analysing a series of images of each animal sequentially captured by the imaging means for determining the at least one of the behaviour, the at least one state or the at least one characteristic of the animal.

155. A data collection system as claimed in claim 153 in which a plurality of identifying means are provided, one identifying means being provided for each animal, and each identifying means comprising data indicative of the identity of the corresponding animal, and the data acquisition means is configured to read the data indicative of the identity of the animals from the respective identifying means, and preferably, each identifying means comprises an electronically readable identity code identifying the corresponding animal, and advantageously, each identifying means comprises a vision readable identity code identifying the corresponding animal, and preferably, the data acquisition means is configured for reading the identity code from each of the identifying means, and advantageously, each identifying means is configured for one of locating externally on and internally in the corresponding animal.

156. A data collection system as claimed in claim 153 in which the primary storing means of the mobile data collection device is configured for storing data indicative of the one of the behaviour, the at least one state or the at least one characteristic of each animal cross-referenced with the identity of the animal, and preferably, the mobile data collection device comprises a location tracking means, and preferably, the location tracking means of the mobile data collection device cooperates with a GPS network in order to identify the location of the mobile data collection device, and advantageously, the mobile data collection device is configured for determining the location of each animal from the location of the mobile data collection device, and preferably, the mobile data collection device is configured to store data indicative of the location of each animal cross-referenced with the data indicative of the identity of the animal in the primary storing means, and advantageously, the primary communicating means of the mobile data collection device is configured to wirelessly communicate with the central server for transmitting the data stored in the primary storing means thereof indicative of the at least one of the behaviour, the at least one state, the at least one characteristic of each animal or the location of each animal cross-referenced with the identity data of the animal, and preferably, the primary communicating means of the mobile data collection device is configured to communicate the data stored in the primary storing means thereof to the central server via a wireless communications network, and advantageously, the primary communicating means of the mobile data collection device is configured to communicate the data stored in the primary storing means thereof to the central server via a GSM network, and preferably, the primary communicating means of the mobile data collection device is configured for communicating the stored data in the primary storing means thereof to the central server just after the collection of the stored data, and advantageously, the primary communicating means of the mobile data collection device is configured for uploading the stored data in the primary storing means thereof to the central server at predefined transmission time intervals, and preferably, the time duration of each predefined transmission time interval lies in the range of 3 hours to 24 hours, and advantageously, the time duration of each predefined transmission time interval lies in the range of 4 hours to 12 hours, and preferably, the time duration of each predefined transmission time interval is approximately 6 hours.

157. A data collection system as claimed in claim 153 in which the data indicative of the at least one of the behaviour, the at least one state or the at least one characteristic of each animal stored in the primary storing means of the mobile data collection device is communicated to the central server by the mobile data collection device in response to receipt of a signal from the central server requesting uploading of the stored data, and preferably, the data indicative of the one of the behaviour, the at least one state or the at least one characteristic of one or more of the animals is communicated to the central server in response to the data indicative of the one of the behaviour, the at least one state or the at least one characteristic being indicative of an emergency state being detected in that of one or more of the animals, and advantageously, the mobile data collection device is powered by an electrical battery, and preferably, the electrical battery of the mobile data collection device comprises a rechargeable battery, and advantageously, the mobile data collection device comprises a solar panel for powering the mobile data collection device and for charging the electrical battery thereof, and preferably, the solar panel of the mobile data collection device comprises a photovoltaic panel, and advantageously, the mobile data collection device is configured to be powered by the prime mover on which the mobile data collection device is mounted, and preferably, the mobile data collection device is configured for mounting on one of the animals.

158. A data collection system as claimed in claim 153 in which the mobile data collection device is configured for mounting on the prime mover, and is configured to be urged past the animals by the prime mover at predefined data collection time intervals, and preferably, the time duration of each predefined data collection time interval lies in the range of 3 hours to 24 hours, and advantageously, the time duration of each predefined data collection time interval lies in the range of 4 hours to 12 hours, and preferably, the time duration of each predefined data collection time interval is approximately 6 hours, and advantageously, when the mobile data collection device is configured for mounting on a prime mover, the primary communicating means is configured for uploading the data stored in the primary storing means indicative of the at least one of the behaviour, the at least one state, the at least one characteristic or the location of each animal cross-referenced with the identity thereof to the central server when the mobile data collection device has returned from each pass of the animals, and preferably, a docking station configured to communicate with the central server is provided and the mobile data collection device is configured to dock in the docking station, and to communicate the data stored in the primary storing means thereof indicative of the at least one of the behaviour, the at least one state, the at least one characteristic or the location of each animal cross-referenced with the identity thereof to the central server via the docking station when the mobile data collection device is docked in the docking station, and advantageously, the mobile data collection device is configured to be mounted on the prime mover so that the mobile data collection device passes within a distance of up to 10 metres from each of the animals, and preferably, the mobile data collection device is configured to be mounted on the prime mover so that the mobile data collection device passes within a distance of up to 7 metres from each of the animals, and advantageously, the mobile data collection device is configured to be mounted on the prime mover so that the mobile data collection device passes within a distance of approximately 3 metres from each of the animals, and preferably, the mobile data collection device is configured for mounting on a prime mover in the form of one of a tractor, a quad-bike and a drone configured for flying, and advantageously, the prime mover comprises an autonomous vehicle.

159. A data collection system as claimed in claim 153 in which a plurality of monitoring devices are provided, one monitoring device being provided for each animal, and being configured for attaching to the corresponding animal, each monitoring device being configured for monitoring the at least one of the behaviour, the at least one state or the at least one characteristic of the corresponding animal, and each monitoring device comprising a secondary storing means for storing data indicative of the monitored one of the at least one of the behaviour, the at least one state or the at least one characteristic of the animal, and preferably, each monitoring device is configured for locating one of externally on and internally in the corresponding animal, and advantageously, the data acquisition means of the mobile data collection device comprises a first secondary communicating means for wirelessly communicating with each of the monitoring devices for receiving the stored monitored data therefrom, and preferably, each monitoring device comprises a second secondary communicating means configured for wirelessly transmitting data stored in the secondary storing means of the monitoring device for reception by the mobile data collection device, and advantageously, the first secondary communicating means of the mobile data collection device is configured for communicating wirelessly with the second secondary communicating means of each monitoring device, and preferably, the first secondary communicating means of the mobile data collection device is configured for communicating wirelessly with the second secondary communicating means of each monitoring device by a radio frequency protocol transmission, and advantageously, the first and second secondary communicating means comprise RFID communicating means, and preferably, the RFID communicating means of each monitoring device comprises a passive RFID communicating means, and advantageously, the second secondary communicating means of each monitoring device is configured for powering by the mobile data collection device, and preferably, the second secondary communicating means of each monitoring device is configured to be powered by electromagnetic induction by a signal transmitted by the first secondary communicating means of the mobile data collection device.

160. A data collection system as claimed in claim 159 in which each monitoring device is configured to transmit data in response to an activating signal from the mobile data collection device, and preferably, each monitoring device is configured to upload data from the secondary storing means thereof to the mobile data collection device through the second secondary communicating means of the monitoring device and the first secondary communicating means of the mobile data collection device, and advantageously, the primary storing means of the mobile data collection device is configured for storing data uploaded from the respective monitoring devices, and preferably, each monitoring device comprises the corresponding one of the identifying means.

161. A data collection system as claimed in claim 159 in which each monitoring device is configured for monitoring the at least one of the behaviour, the at least one state or the at least one characteristic of the corresponding animal during predefined monitoring time periods, and at the end of each predefined monitoring time period, the data captured during that predefined monitoring time period indicative of the at least one of the behaviour, the at least one state or the at least one characteristic of the animal is stored in the secondary storing means, and preferably, the time duration of each predefined monitoring time period lies in the range of 5 minutes to 60 minutes, and advantageously, the time duration of each predefined monitoring time period lies in the range of 10 minutes to 45 minutes, and preferably, the time duration of each predefined monitoring time period is approximately 15 minutes, and advantageously, the predefined monitoring time periods run consecutively one after the other, and preferably, each monitoring device is powered by an electrical battery, and preferably, the electrical battery of each monitoring device comprises a rechargeable battery, and advantageously, the mobile data collection device comprises a smart mobile device, and preferably, the mobile data collection device comprises a smart mobile phone, and advantageously, the animals are remotely located.
